(19) United States
(12) Patent Application Publication
(10) Pub. No.: US 2005/0234305 A1
(43) Pub. Date: Oct. 20, 2005

(54) Medical device and system for providing an image

(76) Inventor: Frederick Licciardi, River Vale, NJ (US)

Correspondence Address: FAY KAPLUN & MARCIN, LLP, 15O BROADWAY, SUITE 702, NEW YORK, NY, 10038, US

(21) Appl. No.: 11/104,799

(22) Filed: Apr. 13, 2005

Related U.S. Application Data
(60) Provisional application No. 60/562,913, filed on Apr. 15, 2004.

(51) Int. Cl. A61B 1/32

(52) U.S. Cl. 600/221

(57) ABSTRACT

Described is a medical device and system for providing an image. The medical device may include a speculum and an imaging device. The speculum is utilized within an orifice of a patient for a surgical procedure and/or a medical examination. The speculum includes a blade which has a distal end which, in an operative position, is inserted into the orifice. The imaging device is mounted on the distal end. When the speculum is inserted into the orifice, the imaging device provides an image of an area within the orifice and surrounding tissues and structures.

### PRIORITY/INCORPORATION BY REFERENCE

The present application claims priority to U.S. Provisional Patent Application No. 60/562,913 entitled “Apparatus and System for Providing an Image” filed on Apr. 15, 2004, the specification of which is expressly incorporated, in its entirety, herein.

### BACKGROUND OF THE INVENTION

Medical examinations and surgical procedures are typically facilitated if a performing physician expands and/or controls an operating field. Often, controlling the field requires one or more medical devices (e.g., a speculum) which are positioned and/or manipulated by the physician. A typical speculum allows the physician to dilate a bodily orifice (e.g, a vagina) to present a relatively larger view of the field therein. For example, in a conventional gynecological examination, the physician may insert the speculum into the vagina of a patient expanding the opening of the vagina. The speculum thus provides an enhanced view of the vagina and structures therein. However, elasticity of tissue comprising the vagina resists the expanding force of the speculum limiting the physician's view thereof. Thus, the physician may utilize several devices to obtain an optimal view of the field and other parts of an internal anatomy (e.g., cervix, uterus, etc.).

In addition to the use of devices such as the speculum, physicians have utilized imaging devices to enhance the view of the field. The imaging devices also have an advantage of providing guidance in conducting the surgical procedure/examination. During the gynecological examination, for example, the physician may insert a scope (e.g., colposcope) into the vagina or position an ultrasound transducer over an abdomino-pelvic region to obtain an image of an inside of the vagina and the structures therein. However, use of both the scope and the speculum may limit movement and/or insertion of further devices (e.g., scalpels, cautery devices, forceps, etc.). That is, the physician and/or personnel assisting the physician must maintain positioning and monitor operation of the speculum, the scope and any other medical devices, while providing enough space for the physician to perform the examination and/or procedure.

### SUMMARY OF THE INVENTION

The present invention relates to a medical device and system for providing an image. The medical device may include a speculum and an imaging device. The speculum is utilized within an orifice of a patient for a surgical procedure and/or a medical examination. The speculum includes a blade which has a distal end which, in an operative position, is inserted into the orifice. The imaging device is mounted on the distal end. When the speculum is inserted into the orifice, the imaging device provides an image of an area within the orifice and surrounding tissues and structures.

### BRIEF DESCRIPTION OF THE DRAWINGS

FIG. 1 shows an exemplary embodiment of a medical device according to the present invention;

FIG. 2A shows an exemplary embodiment of a coupling of an imaging modality to the medical device according to the present invention;

FIG. 2B shows another exemplary embodiment of the coupling of the imaging modality to the device according to the present invention; and

FIG. 3 shows an exemplary embodiment of an imaging system incorporating the medical device according to the present invention.

### DETAILED DESCRIPTION

The present invention may be further understood with reference to the following description and the appended drawings, wherein like elements are provided with the same reference numerals. The present invention is directed to a medical device for providing an image, and a system, incorporating the medical device, accomplishing the same function. More particularly, the medical device includes an imaging modality to provide an image of a field within a patient, while limiting a number of devices inserted into the patient during a medical procedure or examination. As would be understood by those skilled in the art, the field within the patient may include an operable and/or an examined area within an orifice of the patient. The image of the field may be provided to a display to aid a user (e.g., a physician) in manipulating the medical device and/or the image of the field.

In one exemplary embodiment of the present invention, the medical device may include a speculum 13 and the imaging modality (e.g., an ultrasound transducer 14) disposed thereon. In this embodiment, the speculum 13 is used to expand a bodily orifice, naturally occurring (e.g., vagina, anus, mouth, etc.) or unnaturally occurring (e.g., incision, injury, etc.), to provide the user thereof with an increased view of and/or access to an anatomical structure(s) thereinside. For example, the user may insert the speculum 13 into the vagina of the patient to obtain enhanced access to and/or view of structures internal thereto (e.g., cervix, uterus, etc.).

Various embodiments of the present invention may include different medical devices equipped with one or more imaging modalities for use in different medical procedures or examinations. For example, in one exemplary embodiment, an oral surgeon, dentist, orthodontist or otolaryngologist may use the device (e.g., retractor, forceps, insertable cushion, rongeur, dilator, etc.) to expand an oral cavity while performing a procedure or examination (e.g., dental surgery, tooth extraction, jaw surgery, maxillofacial surgery, TMJ surgery, tonsillectomy, adenoidectomy, cyst removal, etc.). The imaging modality may be coupled to the device to allow the user to view the oral cavity and structures therein.

A further exemplary embodiment of the present invention may include a procedure performed on a rectum or an anal cavity. Such procedures may include, but are not limited to, anoscopy, hemorrhoid surgery, rectal prolapse repair, imperforate anus repair, anal fissure repair, cyst removal and other procedures which require or would be facilitated by expansion of the rectum and/or anal cavity. The user may utilize the device (e.g., forceps, dilator, rectal speculum, proctoscope, retractors, etc.) to assist expansion of the anal cavity. The imaging modality may be coupled to the device to allow the physician to view the rectum and/or the anal cavity and structures therein.

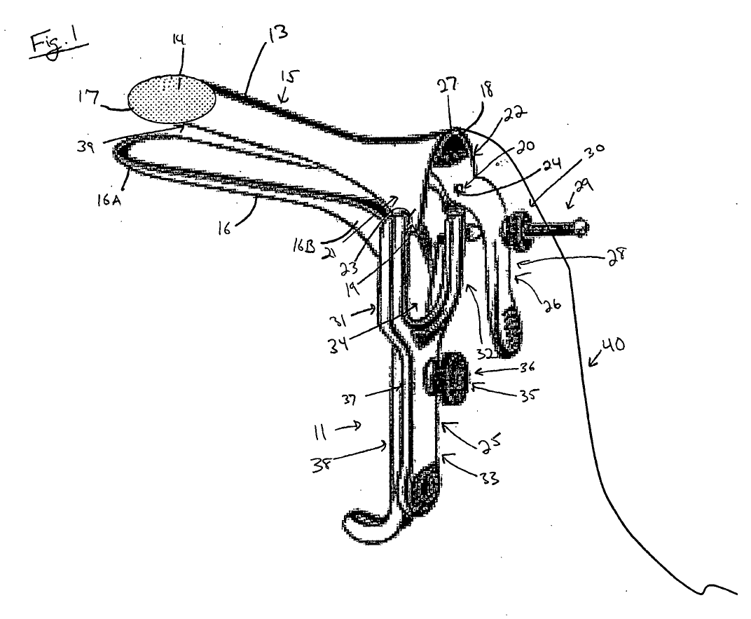

FIG. 1 shows an exemplary embodiment of the speculum 13 used for insertion into a bodily orifice (e.g., vagina, anus, etc.) of the patient, and the imaging modality is the ultrasound transducer 14 used for diagnostic (e.g., imaging) and/or therapeutic ultrasound (e.g., thermal therapy). As stated above, in further exemplary embodiments, the device may be one of a variety of medical devices, such as, for example, a forceps, a weighted speculum, a retractor, etc. Furthermore, those of skill in the art will understand that the imaging modality may be an element which utilizes, alternatively or additionally, a further imaging technique, such as, x-ray, infra-red, etc.

As shown in FIG. 1, the speculum 13 includes a first blade 15 having a distal end 17 and a proximal end 18. The distal end 17 may include a first tip portion which may be rounded to facilitate insertion of the distal end 17 into the orifice. For example, in a gynecological exam, the user inserts the distal end 17 of the first blade 15 into an opening in the vagina projecting the first blade 15 distally into the vagina and spreading a vaginal wall using the first blade 15. The first blade 15 may be configured in a conventional manner or may be shaped as a bar or tube. In either manner, it is preferable to include the rounded tip portion on the distal end 17 of the first blade 15. Those of skill in the art will understand that an embodiment of the speculum 13 including only the first blade 15 may be, for example, a weighted speculum.

The speculum 13 may further include a second blade 16 having a distal end 16A and a proximal end 16B. In this manner, the second blade 16 may have a similar structure to that of the first blade 15, including a second tip portion at the distal 16A. Also, the second blade 16 may be tube- or bar-shaped.

The proximal end 18 of the first blade 15 preferably provides a point of rotation of the first blade 15 relative to the second blade 16. In this manner, the proximal end 18 of the first blade 15 may have a first hole 19 and a second hole 20 disposed on lateral surfaces 21 and 22, respectively. The first hole 19 receives a first peg 23 disposed on a handle 25, and the second hole 20 receives a second peg 24 disposed on the handle 25. When the holes 19 and 20 receive the pegs 23 and 24, respectively, the first blade 15 can rotate relative to the second blade 16 on an axis formed by the pegs 23 and 24 (e.g., radially away from the second blade 16). As is understood by those skilled in the art, the peg and hole combination for rotation of the first blade 15 relative to the second blade 16 may be replaced by several variations, such as, a rod replacing the pegs, convex protrusions that reside in concave valleys, or any other means to allow for rotation of the first blade 15 relative to the second blade 16, or vice-versa.

Movement of the first blade 15 relative to the second blade 16 may be initiated with a depression member 26. The depression member 26 may elevate and/or position the first blade 15 radially away from the second blade 16. In a preferred embodiment, the depression member 26 has a first end 27 attached to the proximal end 17 of the first blade 15 and a second end 28 that can allow the user to push the depression member 26 distally toward the handle 25, thereby rotating the first blade 15 relative to the second blade 16. As is understood by those skilled in the art, any number of elements may be used to move the first blade 15 with respect to the second blade 16 (e.g., a locking gear, a dial, electronic means, etc.). In this embodiment, a user pushes the depression member 26 distally toward the handle 25 to rotate the first blade 15 radially away from the second blade 16. As such, the distal end 17 of the first blade 15 is furthest away from the distal end 16A of the second blade 16 when the depression member 26 is closest to the handle 25.

The depression member 26, and thus, the first blade 15, may be maintained in a static position. As the user inserts the speculum 13 into the orifice and presses the depression member 26 distally towards the handle 25, the first blade 15 and the second blade 16 contact the tissue comprising the orifice and expand an opening thereof and/or tissue therein. The user may then lock the depression member 26 in a desired position by, for example, utilizing a first locking arrangement. The first locking arrangement may include moving a nut 30 distally along a rod 29 attached to either the handle 25 or the proximal end 16B of the second blade 16. As understood by those of skill in the art, the rod 29 is preferably threaded, thereby allowing the nut 30 to ride therealong both proximally and distally.

The rod 29 has a distal end attached to the handle 25 and passes along a side of or through a hole in the depression member 26 to a free proximal end. After the nut 30 comes in contact with the depression member 26 and moves further distally along the rod 29, the first blade 15 is forced radially away from the second blade 16. In operation, after the speculum 13 has been inserted into the orifice, the user moves the nut 30 distally along the rod member 29 into a predetermined position. Once in the predetermined position, the nut 30 preferably acts as a backstop, preventing the depression member 26 from moving proximally therepast. As a result, the first blade 15 may be maintained in a static position with respect to the second blade 16 due to the depression member 26 locked in position by the nut 30, and under a compressive force on the first blade 15 and the second blade 16 provided by the tissue which the blades come in contact with.

The handle 25 of the speculum 13 is preferably disposed in a substantially perpendicular position relative to the first blade 15 and the second blade 16. As understood by those of skill in the art, the handle 25 may be ergonomically designed in any manner which would facilitate manipulation and control by the user. In one exemplary embodiment, the handle 25 is Y-shaped having a first arm 31 and a second arm 32, each extending from a base 33. The first arm 31 may have the first peg 23 located on an upper portion thereof, and the second arm 32 may have the second peg 24 located on an upper portion thereof. As described above, the first and second pegs 23, 24 provide a point of attachment and rotation for the first blade 15.

An aperture 34, defined by a space between the first arm 31, the second arm 32, the base 33 and the first blade 15, may provide a view into the orifice after the speculum 13 has been inserted and the first blade 15 has been rotated away from the second blade 16. In this manner, it is contemplated that depression member 26 may be off-set from a longitudinal axis of the first blade 15, thereby fully exposing the aperture 34 to the user. Furthermore, in a preferred embodiment, the proximal end 18 of the first blade 15 is shaped such that the aperture 34 does not substantially decrease in area when the first blade 15 is rotated away from the second blade 16.

The base 33 of the handle 25 may be equipped with a second locking arrangement 35 including a control element (e.g., a knob 36) and an engagement element (e.g., a screw 37) which passes through a bore (e.g., a threaded bore) in the handle 25. A distal end of the screw 37 contacts a slide member 38 which is connected to the proximal end 16B of the second blade 26. When the knob 36 is turned in a first direction, the distal end of the screw 37 moves distally within the bore until it contacts the slide member 38. Further distal advancement of the screw 37 essentially prevents the slide member 38 from moving vertically relative to the handle 25 by creating a frictional hold. Turning the knob 36 in a second direction releases the screw 37 from the slide member 38, thus, allowing the user to move the slide member 28 vertically relative to the handle 25 changing a distance between the first blade 15 and the second blade 16. Movement of the slide member 28 relative to the handle 25 may increase or decrease an area of the aperture 34. As understood by those of skill in the art, the engagement element may be any component (e.g., a locking gear, a clip, a buckle, etc.) which would provide for temporary static positioning of the slide member 28 relative to the handle 25.

As shown in FIG. 1, the ultrasound transducer 14 is coupled to the distal end 17 of the first blade 15. As understood by those of skill in the art, the transducer 14 may be coupled to the first blade 15 in any manner, temporary (e.g., detachable) or permanent, including, but not limited to, embedded, mechanically/chemically bonded, frictional fit, detachably clipped, etc. The transducer 14 may be positioned such that, upon activation, it provides an image of the field around the first blade 15. In this manner, the transducer 14 may be mounted to an inner surface 39 of the first blade 15. In another embodiment, the transducer 14 is affixed, on an angle, to the tip portion of the first blade 15. As such, the image provided thereby may project distally of the speculum 13. Those of skill in the art will understand that the transducer 14 may be positioned, oriented and/or configured in any manner which would provide to a user thereof the image desired.

In another exemplary embodiment, the a further transducer (not shown) may be coupled to the second blade 16 in a similar manner as described for the transducer 14. Specifically, the further transducer may be positioned on an inner surface of the distal end 16A of the second blade 16. As understood by those of skill in the art, two or more transducers may be used to create a three-dimensional image by merging a series of signals from the transducers. Furthermore, use of more than one transducer 14 may expand the image of the field within the orifice.

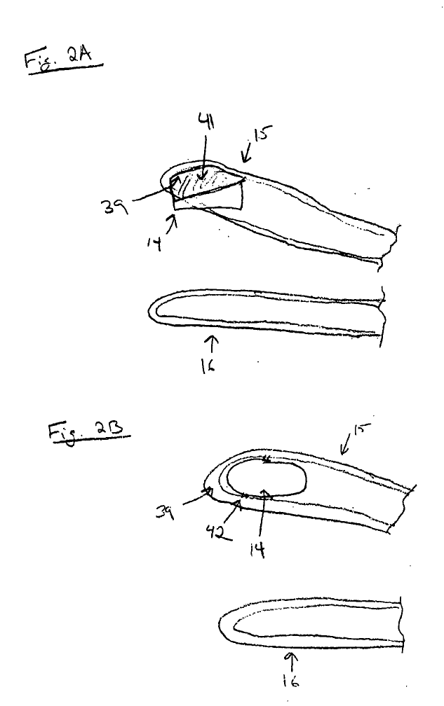

Further exemplary embodiments for coupling the transducer 14 to the first blade 15 are seen in FIGS. 2A and 2B. Although, FIGS. 2A and 2B depict mechanical mechanisms for directing ultrasound energy in one or more directions from the transducer 14, those skilled in the art will understand that the ultrasound energy may be focused in other manners and in any number of directions depending upon the field that the user wishes to visualize. Specifically, the user of the speculum 13 may adjust an amplitude and a frequency of the ultrasound energy to manipulate the image provided by the transducer 14.

In FIG. 2A, the transducer 14 may be coupled to an angling block 41, which is attached to the inner surface 39 of the first blade 15. The angling block 41 may be positioned to allow the transducer 14 to direct ultrasound energy in a predetermined direction (e.g., perpendicular to the second blade 16) reducing any error in directional focus of the ultrasound energy due to rotation of the first blade 15, as when the transducer 14 is immovably implanted in the first blade 15. The angling block 41 may be any solid or semi-solid object temporarily or permanently bonded to the inside surface of the first blade 15. Those of skill in the art will understand that the angling block 41 may be attached to the first blade 15 in any manner, such as, for example, embedded, fastened, chemically bonded, welded, etc.

In FIG. 2B, the transducer 14 is movably attached to the first blade 15, whereby the transducer 14 can focus ultrasound energy in any user-desired direction. Movement of the transducer 14 may be accomplished via a pin 42 inserted through the transducer 14 that allows the transducer 14 to rotate in a free (e.g., gravitationally) or controlled manner relative to the first blade 15. As is understood by those skilled in the art, rotation of the transducer 14 may be controlled mechanically and/or electronically. That is, a drive system may be used to angle the transducer 14 relative to the first blade 15. In one embodiment, the drive system includes a small motor and a gearing arrangement attached to the transducer 14 and/or the speculum 13. The user may control the drive system to move the transducer 14 through a predefined range of motion (e.g., 180 degrees) relative to the first blade 15. In another embodiment, the transducer 14 may rotate along any axis thereof relative to the first blade 15. Those of skill in the art will understand that the embodiments and mechanisms depicted in FIGS. 2A and 2B may be utilized with respect to any further transducer(s) attached to the speculum 13.

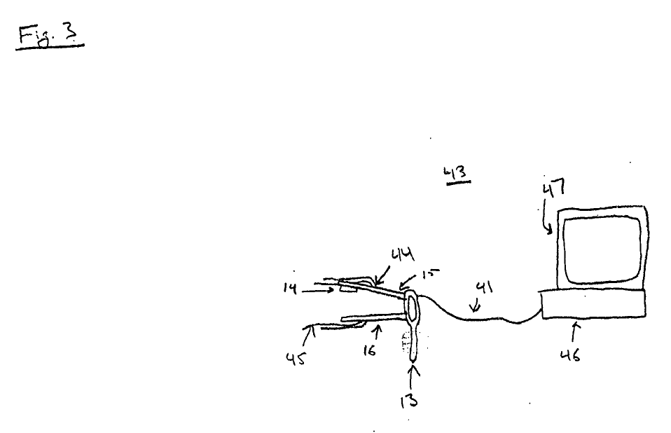

FIG. 3 shows an exemplary embodiment of a system 43 according to the present invention. In operation, the speculum 13 is inserted into an orifice 44 on a patient. As would be understood by those of ordinary skill in the art, the present invention may be used for any bodily orifice (e.g., naturally or non-naturally occurring) and for any device which may be used to visualize the orifice. The speculum 13 is inserted into the orifice 44 in a closed position. In the closed position, the first blade 15 is preferably in contact with the second blade 16. That is, in the close position, no force has been applied to the depression member 26 to rotate the first blade 15 relative to the second blade 16. Preferably, the distal end 17 of the first blade 15 is flush against the distal end 16A of the second blade 16. In this manner, the distal ends 17, 16A present a rounded, conical tip. As understood by those of skill in the art, insertion of the speculum 13 into the orifice 44 may be facilitated by applying a lubricant thereto.

After insertion into the orifice 44, the user may, manually or electro-mechanically, rotate the first blade 15 away from the second blade 16 into an open position. In a further embodiment, prior to or after the speculum 13 is in the open position, the user may release the slide member 38 from the screw 37, move the slide member 28 relative to the handle 25, thus adjusting the distance between the first blade 15 and the second blade 16 into an expanded position. Although the adjustment of the distance between the first blade 15 and the second blade 16 is termed the expanded position, those of skill in the art will understand that decreasing the distance between the first blade 15 and the second blade 16 via the slide member 38 may result in a contracted position. In the gynecological procedure, for example, the user inserts the speculum 13 into the vagina in the closed position. Then, the user may move the speculum 13 into the expanded (or contracted) position by releasing and moving the slide member 38. In the expanded position, the area of the aperture 34 increases allowing the user to have a greater viewing field between the first blade 15 and the second blade 16.

In the open position, the first blade 15 and/or the second blade 16 may contact an inner wall 45 of the orifice 44. For example, in the gynecological procedure, the inner wall 45 may be a vaginal wall, whereas in a user-created incision, the inner wall 45 may be a portion of the incision or a body organ/tissue into which the speculum 13 is inserted. In either instance, the inner wall 45 may have an elastic characteristic which allows a predetermined amount of expansion thereof in response to an expansive force applied by the first blade 15 and the second blade 16. That is, expansion past the predetermined amount may cause structural damage to the organ/tissue which is undesirable. Thus, the user may utilize the first locking arrangement by pressing the depression member 26 distally, followed by moving the nut 30 distally along the rod 29. When the user feels a resistive force on the depression member 26 (e.g., inner wall 45 has reached or substantially reached the predetermined amount of expansion), the nut 30 may be moved distally along the rod 29 until it contacts the depression member 26. Thus, the first blade 15 remains statically positioned relative to the second blade 16 while inside the orifice 44.

At any point during the procedure/examination, the user may activate the transducer 14. For example, the user may activate the transducer 14 to facilitate insertion and/or guidance of the speculum within the orifice 44. The transducer 14 may be activated when the speculum 13 is in the open position and/or the expanded position while in the orifice 44. Thus, those of skill in the art will understand that activation of the transducer 14 may occur prior to, during or after insertion of the speculum 13, as well as prior to, during of after completion of the procedure/examination. Furthermore, the transducer 14 may be connected to a processing arrangement 46 (e.g., an ultrasound device, a computer, etc.) via a wired connection 40 or a wireless connection. In either embodiment, the speculum 13 and/or the procession arrangement may include a controller (e.g., switch, button, dial, etc.) for activating the transducer 14. Preferably, the controller remains outside of the orifice 44 when the speculum 13 has been inserted thereinto.

When the transducer .14 is activated, ultrasound energy transmitted and subsequently received by the transducer 14 is converted to data which is transmitted to the processing arrangement 46. That is, the processing arrangement 46 may provide power to the transducer 14 which generates the ultrasound energy used to create the image of the field. As shown in the embodiment of FIG. 3, the transducer 14 transmits the ultrasound energy distally therefrom (e.g., toward the second blade 16) and generates the image based on the data (e.g., refractions, reflections and scattering of the ultrasound energy received by the transducer 14). The data is transmitted to the processing arrangement 46 via the wire 41 or a wireless transmission.

The processing arrangement 46, upon receipt of the data, generates the image and provides it on a display 47. According to the present invention, the data may provide for a two-dimensional image or a three-dimensional image which is shown on the display 47. Further, the data may be transmitted to the processing arrangement 46 continuously, so that the display 47 shows a dynamic image. Alternatively, the data may be transmitted and/or the image may be generated at a predetermined interval, so that the display 47 shows a plurality of static images successively. Viewing the display 47, the user may manipulate the speculum 13, the first blade 15 and/or the transducer 14 to manipulate the image provided thereby. Alternatively, the user may control the image via the controller on the processing arrangement 46.

The present invention has been described with reference to specific exemplary embodiments thereof. It will, however, be evident that various modifications and changes may be made thereto without departing from the broadest spirit and scope of the present invention as set forth in the disclosure herein. Accordingly, the specification and drawings are to be regarded in an illustrative rather than restrictive sense.

1. An apparatus, comprising:
a speculum utilized within an orifice of a patient for at least one of a surgical procedure and a medical examination, the speculum including a blade, the blade having a distal end which, in an operative position, is inserted into the orifice; and
an imaging device attached to the distal end,
wherein when the speculum is inserted into the orifice, the imaging device provides an image of an area within the orifice.

2. The apparatus of claim 1, wherein the speculum includes a further blade, the further blade having a further distal end which, in an operative position, is inserted into the orifice.

3. The apparatus of claim 2, wherein the speculum includes a further imaging device attached to the further blade.

4. The apparatus of claim 1, wherein the imaging device uses one of ultrasound energy, x-ray energy and infra-red energy to provide the image.

5. The apparatus of claim 1, wherein the speculum is one of a forceps, a retractor and a weighted speculum.

6. The apparatus of claim 1, wherein the orifice is one of a naturally occurring and an unnaturally occurring bodily orifice.

7. The apparatus of claim 1, wherein the imaging device is an ultrasound transducer.

8. The apparatus of claim 1, wherein the imaging device is one of embedded in, bonded to and clipped to the blade.

9. The apparatus of claim 1, wherein the imaging device is movable relative to the blade.

10. The apparatus of claim 1, wherein the blade is one of a tube and a bar.

11. The apparatus of claim 1, further comprising:
a control element connected to the imaging device for adjusting the image provided thereby.

12. The apparatus of claim 2, wherein the speculum includes at least one further imaging device attached to one of the blade and the further blade.

13. A system, comprising:
a speculum utilized within an orifice of a patient for at least one of a surgical procedure and a medical examination, the speculum including a blade, the blade having a distal end which, in an operative position, is inserted into the orifice;
an imaging device attached to the distal end, the imaging device providing an image of an area within the orifice when the speculum is inserted into the orifice;
a processing arrangement receiving the image from the imaging device; and
an output arrangement coupled to the processing arrangement and outputting the image from the imaging device.

14. The system of claim 13, wherein the speculum includes a further blade, the further blade having a further distal end which, in an operative position, is inserted into the orifice.

15. The system of claim 14, wherein the speculum includes a further imaging device attached to the further blade.

16. The system of claim 13, wherein the imaging device uses one of ultrasound energy, x-ray energy and infra-red energy to provide the image.

17. The system of claim 13, wherein the speculum is one of a forceps, a retractor and a weighted speculum.

18. The system of claim 13, wherein the output arrangement is a display device displaying the image.

19. The system of claim 13, wherein the imaging device is an ultrasound transducer.

20. The system of claim 13, wherein the imaging device is one of embedded in, bonded to and clipped to the blade.

21. The system of claim 13, wherein the imaging device is movable relative to the blade.

22. The system of claim 13, wherein the blade is one of a tube and a bar.

23. The system of claim 14, wherein the speculum includes at least one further imaging device attached to one of the blade and the further blade.
